(19) United States
(12) Patent Application Publication
(10) Pub. No.: US 2011/0034548 A1
(43) Pub. Date: Feb. 10, 2011

(54) Method for Suspending a Flavonoid in a Beverage

(75) Inventors: Nicole Milsap, Walnut Creek, CA (US); Jia Khiun Wee, Palatine, IL (US); Vincent E.A. Rinaldi, Bethel, CT (US); Craig Hutson, Mundelein, IL (US); Timothy J. Gallagher, Chicago, IL (US); Steven E. Havlik, Crystal Lake, IL (US); Zeinab Ali, Crystal Lake, IL (US)

Correspondence Address: BANNER & WITCOFF, LTD.;and ATTORNEYS FOR CLIENT NO. 006943, 10 SOUTH WACKER DR., SUITE 3000, CHICAGO, IL, 60606, US

(73) Assignee: Stokely-Van Camp, Inc., Chicago, IL (US)

(21) Appl. No.: 12/853,384

(22) Filed: Aug. 10, 2010

Related U.S. Application Data
(60) Provisional application No. 61/232,612, filed on Aug. 10, 2009.

(51) Int. Cl. A61K 31/352; A61P 3/02

(52) U.S. Cl. 514/456

(57) ABSTRACT

Disclosed is a method for suspending microparticulated flavonoid in a beverage by incorporating a microparticulated flavonoid and at least one suspension agent into a beverage. A composition comprising microparticulated quercetin and a suspension agent is also disclosed.

### CROSS-REFERENCE TO RELATED APPLICATIONS

This application claims priority to U.S. Provisional Patent Application No. 61/232,612 filed on Aug. 10, 2009, the disclosure of which is expressly incorporated herein by reference.

### FIELD OF THE INVENTION

The present invention relates generally to a method for suspending quercetin in a liquid medium and a composition containing quercetin and a suspension agent.

### BACKGROUND

Flavonols, in general, are used as nutritional supplements to provide, for example, antioxidants. Quercetin, in particular, is considered a powerful antioxidant. Quercetin is a natural, plant-derived, flavonoid. In particular, quercetin is the aglycone form of a number of other flavonoid glycosides, such as rutin and quercitrin, found in citrus fruit, cranberries, blueberries, buckwheat, onions, and other vegetables, fruits, and green plants. The chemical structure of quercetin is illustrated below:

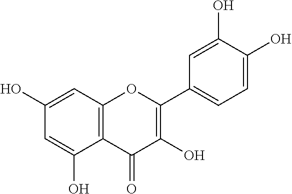

### 2-(3,4-dihydroxyphenyl)-3,5,7-trihydroxy-4H-chromen-4-one

It is desired to use quercetin as a nutritional supplement in food products such as beverages. However, quercetin, which is typically obtained as a powder, is insoluble in water and therefore difficult to disperse and mix into products. When added to liquid media, quercetin usually agglomerizes and settles to the bottom of the beverage, thereby resulting in a product that is not visually appealing to the consumer.

Therefore, a need exists in the food and beverage industry to provide the consumer with a food product containing quercetin wherein the quercetin is easily dispersed and suspended in the product.

### BRIEF SUMMARY

The present invention relates to a method for suspending quercetin particles in a beverage and a composition for a beverage containing microparticulated quercetin. It was discovered that incorporating microparticulated quercetin into a beverage along with a suspension agent results in a visually acceptable quercetin-supplemented product with improved mouthfeel, drinkability and visual appearance.

### DETAILED DESCRIPTION OF THE INVENTION

The present invention relates to a method for suspending quercetin particles in a liquid medium, such as a beverage, and a composition containing quercetin particles and at least one suspension agent.

In one aspect of the present invention, a method for suspending quercetin in a beverage includes introducing to the beverage at least one suspension agent and microparticulated quercetin, wherein the suspension agent is present in an amount sufficient to suspend the microparticulated quercetin in the beverage. “Microparticulated” or “microparticulate” as used in the instant application means a small particle ranging in size from about 0.1 um to about 50 um with an average particle size below about 10 um.

The beverage may be any suitable beverage including, but not limited to, juices, carbonated soft drinks, water, dairy and isotonic beverages. One of ordinary skill in the art of the chemical and food sciences would recognize that any flavonol may be used in accordance with the present invention.

The quercetin added to the beverage is microparticulated. The microparticulated quercetin in the homogenized slurry has an average particle size less than 10 microns, in particular less than 3 microns or less than 1 micron. For example, at least 95% of the particles have a particle size less than 20 microns and 80% of the particles have a particle size less than 3 microns. In another aspect, at least 95% of the particles have a particle size less than 10 microns and at least 80% of the particles have a particle size less than 1 micron.

The at least one suspension agent may be a gum, starch, or pectin selected from, but not limited to, the following: guar gum, xanthan gum, gellan gum, acacia gum, modified food starch, propylene glycol alginate, or combinations of the aforementioned ingredients.

The suspension agent is present in the beverage in a range of about 0.005-0.5 wt % of the beverage. The suspension agent is added to the beverage in an amount sufficient to suspend the quercetin in the solution. For example, about 0.01 to about 3 grams, for instance about 0.2 grams of the suspension agent may be added to suspend about 0.5-1.5 grams, for instance about 0.5 grams of quercetin in about 80-1000 milliliters, for instance about 500 milliliters of solution. If the suspension agent selected is gellan gum, then about 0.01-0.6 grams, such as about 0.05-0.3 grams, of gellan gum may be included in the beverage. The inventors discovered that the length of time that the quercetin stays suspended in the liquid media varies depending on the type(s) of suspension agent(s) used. For example, the quercetin can stay suspended in a beverage for about 12 hours to about six weeks or longer, depending on the suspension agent(s) used.

The applicants of the instant invention also discovered that although the beverage appeared relatively viscous and/or thick upon adding the quercetin to the beverage, by using the quercetin in conjunction with the suspension agent, as described in this application, the beverage has a thin consistency upon consumption. This unexpected result, the thin consistency notwithstanding the viscous visual appearance, is also advantageous, as it leads to consumer likability and acceptance of the beverage.

In another aspect of the present invention, the microparticulated quercetin is added to the beverage in the form of a quercetin slurry. Dry quercetin can be added to water and homogenized to form a quercetin slurry. Food-grade preservatives and acidulants may also be added to the slurry. Incorporating quercetin into the beverage via the quercetin slurry results in improved suspension and dispersibility. It was further discovered that by incorporating the quercetin slurry into the beverage, along with the suspension agent, that low concentration of the suspension agent may be added with a high concentration of quercetin, and even though the quercetin is present in high concentrations and the beverage may look viscous, upon consumption it tastes thin and is easily ingested by the consumer.

The method of the present invention may also include one or more additional ingredients selected from the group consisting of carbohydrates, salts, salt blends, food-grade acids, flavors, colors, Vitamin B3, Vitamin C, non-nutritive and/or nutritive sweeteners and combinations of these ingredients.

The method described herein is advantageous as it successfully suspends quercetin in the beverage and thereby deters settling of the quercetin to the bottom of the beverage's packaging.

The present invention also relates to compositions comprising quercetin and at least one suspension agent. In one aspect of this invention, about 95% of the quercetin is below about 10 microns and the at least one suspension agent is present in an amount sufficient to suspend the quercetin in a liquid medium.

Examples of the present invention is as follows:

### Example 1

The quercetin slurry is prepared by adding quercetin (powder form) to water, adding citric acid and potassium sorbate and homogenizing. The components are present in the following concentrations in the slurry:

| | Amount |
|---|---|
| Ingredient | (% by wt.) |
| Water | 89-99% |
| Quercetin | 1-10% |
| Citric Acid | 0.1-0.4% |
| Potassium Sorbate | 0.03-0.12% |
| Total | 100.00% |

The quercetin slurry is added to the beverage and suspended in the beverage using gellan gum. Additional ingredients are added in the concentrations (w/w) listed below to make an isotonic beverage containing quercetin. The pH range of the resultant isotonic beverage may be about 2.5-4.5, such as about 3.5.

| | Amount |
|---|---|
| Ingredient | (% by wt.) |
| Water | 60-96% |
| Liquid Sucrose | 1-24% |
| Salt Blend | 0.01-0.50% |
| Quercetin Slurry | 0.9-30.0% |
| Gellan gum | 0.010-0.08% |
| Citric Acid | 0.05-0.50% |
| Mango Flavor | 0.05-0.30% |
| Yellow #6 Color 10% solution | 0.01-0.40% |
| Liq. Sucralose (25%) | 0.001-0.05% |
| Ace K | 0.0005-0.003% |
| Vitamin C (Ascorbic Acid) | 0.01-1.2% |
| Vitamin B3 (Niacinamide) | 0.004-0.04% |

### Example 2

In another example of the present invention, the quercetin slurry is prepared using the same method as described in Example 1. A gellan gum is added to the slurry in the concentrations set forth below. Additional ingredients, including a high intensity natural sweetener are added to create a zero calorie orange-flavored isotonic beverage. The pH of the resultant beverage is about 3.5.

| | Amount |
|---|---|
| Ingredient | (% by wt.) |
| Water | 95% |
| Citric Acid | 0.050-0.30% |
| Phosphoric Acid | 0.01-0.20% |
| Vitamin B3 | 0.001-0.015% |
| Ascorbic Acid | 0.05-0.35% |
| Gellan gum | 0.015-0.06% |
| Quercetin slurry | 1-8% |
| Salt Blend | 0.10-0.5% |
| Reb A | 0.010-0.035% |
| Orange Flavor | 0.050-0.30% |

Alternatively, or in addition to, the salt blend listed above, the beverage may contain sea salt to produce a zero-calorie isotonic beverage.

Below is yet another example of the present invention using the quercetin slurry prepared using the method described in Example 1:

| | Amount |
|---|---|
| Ingredient | (% by wt.) |
| Water | 60-99% |
| Sucrose (dry) | 1-8% |
| Quercetin Slurry (5.2%) | 0.9-30% |
| Gellan Gum | 0.01-0.20% |
| Citric Acid | 0.05-0.5% |
| Flavor | 0.05-0.3% |
| Color | 0.01-0.4% |
| Stevia | 0.0010-0.0500% |
| Erythritol | 0.1-2.5% |
| Vitamin C | 0.01-1.5% |
| Vitamin B3 | 0.001-0.5% |

In yet another example of the present invention, the quercetin slurry is prepared using the same method as described in Example 1.

| | Amount |
|---|---|
| Ingredient | (% by wt.) |
| Water | 60-99% |
| Sucrose | 3-25% |
| Salt Blend | 0.01-0.50% |
| Quercetin Slurry (5.2%) | 3-30% |
| Gellan gum | 0.01-1.0% |
| Flavor | 0.05-30% |
| Color | 0.01-0.40% |
| Vitamin C | 0.25-1.0% |
| Vitamin B3 | 0.01-0.04% |

This invention may be embodied in other specific forms without departing from the spirit or essential characteristics thereof. The foregoing embodiments, therefore, are to be considered in all respects illustrative rather than limiting the invention described herein. The scope of the invention is thus indicated by the appended claims, rather than by the foregoing description, and all changes that come within the meaning and range of equivalency of the claims are intended to be embraced therein.

We claim:

1. A method for suspending microparticulated quercetin particles in a beverage comprising the steps of a) introducing at least one suspension agent into a beverage; and b) introducing microparticulated quercetin into the beverage, wherein the suspension agent is present in an amount sufficient to suspend the microparticulated quercetin in the beverage.

2. The method of claim 1 wherein the at least suspension agent is a gum selected from the group consisting of guar gum, xanthan gum, gellan gum, pectin, and combinations thereof.

3. The method of claim 1 wherein the beverage is an isotonic beverage.

4. The method of claim 1 wherein the beverage also includes at least one additional ingredient selected from the group consisting of carbohydrates, salts, salt blends, food-grade acids, flavors, colors, Vitamin B3, Vitamin C, sweeteners and combinations thereof.

5. The method of claim 4 wherein salt is present in the beverage in a concentration of about 0.1-0.5 wt % based on the total weight of the beverage and carbohydrate is added to the beverage in a concentration of about 1-25 wt % based on the total weight of the beverage.

6. The method of claim 5 wherein carbohydrate is added to the beverage in a concentration of about 1-3 wt % based on the total weight of the beverage.

7. The method of claim 4 wherein the beverage also contains at least one salt and at least one sweetener selected from the group consisting of sucralose, acesulfame potassium, stevia, monatin, luo han guo, mogrosides, diterpene, triterpene glycosides and combinations thereof.

8. A method for suspending quercetin particles in a beverage comprising combining at least one suspension agent and microparticulated quercetin, wherein the suspension agent is present in an amount sufficient to suspend the microparticulated quercetin in the beverage, and wherein the microparticulated quercetin is present in the form of a quercetin slurry comprising quercetin, water, at least one food grade acid and at least one food grade preservative.

9. The method of claim 8 wherein the suspension agent is a gum selected from the group consisting of guar gum, gellan gum, xanthan gum, pectin, and combinations thereof.

10. The method of claim 9 wherein the gum is present in the beverage in a concentration of about 0.005-0.06 wt % based on total weight of the beverage.

11. The method of claim 10 wherein the gum is present in the beverage in a concentration of about 0.02 wt % based on total weight of the beverage.

12. The method of claim 8 wherein the beverage has a pH of about 2.5-4.5.

13. The method of claim 12 wherein the pH of the beverage is about 3.2-3.9.

14. The method of claim 8 wherein the at least one food-grade acid is citric acid.

15. The method of claim 8 wherein the at least one food grade preservative is potassium sorbate.

16. A composition for a comestible comprising quercetin and at least one suspension agent, wherein about 95% of the quercetin has a particle size below about 10 microns and the at least one suspension agent is present in an amount sufficient to suspend the quercetin in a liquid medium.

17. A composition for a comestible comprising microparticulated quercetin and at least one suspension agent, wherein the at least one suspension agent is present in an amount sufficient to suspend the microparticulated quercetin in a liquid medium, and wherein the microparticulated quercetin is present in the form of a quercetin slurry comprising quercetin, water, at least one food grade acid and at least one food grade preservative.

18. The composition of claim 14 wherein the comestible includes at least one salt and at least one carbohydrate.

19. A method for enhancing endurance during a physical activity comprising administering to a subject in need thereof an effective amount of a composition comprising quercetin, a stabilizing agent, at least one salt and at least one carbohydrate.
